(19) United States
(12) Patent Application Publication
(10) Pub. No.: US 2010/0331675 A1
(43) Pub. Date: Dec. 30, 2010

(54) PROCESS FOR ACQUIRING A THREE-DIMENSIONAL RADIOLOGICAL IMAGE OF AN ORGAN IN MOVEMENT

(76) Inventors: Francois Kotian, Villepreux (FR); Regis Vaillant, Villebon Sur Yvette (FR); Elisabeth Soubelet, Chattarpur Farms Chattarpur (FR)

Correspondence Address: General Electric Company;GE Global Patent Operation, 2 Corporate Drive, Suite 648, Shelton, CT, 06484, US

(21) Appl. No.: 12/741,660

(22) PCT Filed: Aug. 29, 2008
(86) PCT No.: PCT/US08/74707

§ 371 Date: Sep. 15, 2010

(30) Foreign Application Priority Data
Nov. 6, 2007 (FR) FR 0758824

(51) Int. Cl. A61B 6/00; G06T 7/00

(52) U.S. Cl. 600/431; 382/132; 382/154

(57) ABSTRACT

An embodiment of the invention relates to a process for acquiring a three-dimensional radiological image of an organ in movement of a patient, according to which a control unit executes steps of: (53, 54) receiving a signal representative of a movement parameter of the organ, (53, 54) detecting variation in the signal due to artificial maintenance of the organ in a reduced state of movement, and in response to detection, (59, 510) triggering acquisition of a sequence of images by a radiological imaging device to reconstruct a three-dimensional radiological image of the organ from the sequence of images.

### BACKGROUND OF THE INVENTION

1. Field of the Invention

The field of the invention relates to a process for acquiring a three-dimensional image of an organ in movement, such as a heart, for example.

2. Description of Prior Art

Non-surgical cardiac intervention procedures are generally carried out in catheterisation wards (known as <<cath labs>>) under X-ray guidance imaging. Radiological systems used for these procedures offer a variety of modes of dynamic imagery which will be designated, for the sake of clarity, by the term fluoroscopy.

More and more often, cardiologists refer to three-dimensional images, such as X-ray tomodensitometer (“scanner”) or MRI (Magnetic Resonance Imaging), acquired prior to intervention (during a diagnostic examination), to display the anatomy of the heart during intervention. This provides a complement of significant anatomical information, such as fluoroscopy, even though, producing a dynamic image in real time during the procedure, it does not naturally display correctly. In particular this imagery is protective, therefore giving no in-depth information, and dependent on an iodised contrast product for displaying vascular structures of interest.

For example, pre-acquired tomographic images are generally utilised in structural cardiac procedures (replacement of an aortic valve, regeneration of the myocardium) or in rythmology procedures (thermal ablation for treatment of auricular fibrillation). The pre-acquired three-dimensional image and the real-time fluoroscopic image are displayed side by side or in the form of a single composite image obtained by fusion of the three-dimensional image and of the fluoroscopic image.

Yet, in certain cases, it is not possible for the cardiologist to refer to a pre-acquired three-dimensional image.

In fact, it can eventuate that no three-dimensional image is available at the time of intervention (for example, the image has not yet been transferred to the cath lab or no imaging examination has been conducted).

In addition, even if a tomographic image is available, this image can be unusable by the cardiologist. In certain cases, the three-dimensional image has incoherencies with the real-time fluoroscopic image. These incoherencies are due to the fact that, even though from the same patient, these images can correspond to a different anatomical or physiological reality, making it impossible to establish correspondence between the images.

In parallel, certain interventions require place the heart to be placed artificially in a state of reduced movement. This can be accomplished by rapid electrical stimulation equipment (typically at a rhythm of 150 to 220 beats per minute) by means of a cardiac stimulator or by injection of an electrophysiological substance causing asystolis, or a cardiac blockage composition (such as a solution of adenosine triphosphate).

### SUMMARY OF THE INVENTION

An aim of an embodiment of the invention is to allow acquisition of a good-quality three-dimensional image using fluoroscopy equipment.

This problem is resolved within the scope of the present invention by an embodiment of a process for acquiring a three-dimensional radiological image of an organ in movement. In this process, a control unit executes the steps of:

receiving a signal representative of a movement parameter of the organ;

detecting a variation in the signal due to artificially keeping the organ in a reduced state of movement; and

in response to the detection, triggering acquisition of a sequence of images by a radiological imaging device in light of reconstructing a three-dimensional radiological image of the organ from the sequence of images.

The process described employs equipment intended for maintaining the organ in a reduced state of movement: such equipment is, in certain cases, already installed on the patient with a view to intervention.

The control unit synchronises acquisition of the image sequence with keeping the organ in a reduced state of movement of the organ.

On one hand, this effectively limits the dose of X-rays received by the patient.

On the other hand, this effectively secures the acquisition process by automating triggering of the acquisition. The duration of artificial maintenance of the organ in a reduced state of movement can be limited to a minimum necessary for acquisition.

The detection step may comprise comparison of the received signal with an expected reference signal indicative of the state of reduced movement of the organ.

The process can also comprise a step of:

controlling the maintenance of the organ in a reduced state of movement.

The maintenance of the organ in a reduced state of movement is done by rapid cardiac stimulation or by injection of a blocking solution.

The process can comprise a step of:

controlling the stopping of a ventilation device of the patient prior to triggering the acquisition step.

So, the process also synchronises stopping of the ventilation device with acquisition of the sequence of images. The stopping of the ventilation device actually likewise reduces the movements of the organ which would be due to the movements of the thoracic cage of the patient, the stopping duration of the ventilation having naturally to be reduced to the minimum necessary for the acquisition of images.

The process can further comprise steps of:

controlling an injection of a contrast product; and

timing a period for diffusion of the contrast product and triggering acquisition of a sequence of images once the interval has passed.

The process can further comprise steps of:

monitoring evolution of physiological parameters of the patient,

controlling interruption of the process in case of detection of abnormal evolution of one of the physiological parameters.

Therefore, in the event of anomaly, the process is automatically terminated.

The process can likewise comprise steps of:

detecting pressure exerted on a triggering activator by an operator,

controlling interruption of the process in the event of relinquishing the pressure exerted by the operator.

Therefore, the operator can decide to terminate the process at any time.

In particular, interruption of the process comprises ceasing of the maintenance of the organ in a reduced state of movement.

Embodiments of the invention also relate to a control unit of an acquisition device of a three-dimensional radiological image, the unit being suitable for receiving at the input a signal representative of a movement parameter of an organ and generating at the output a signal for controlling the image acquisition device, the unit being programmed for executing the steps of the process described above.

Another embodiment of the invention finally relates to a radiological imaging device comprising such a control unit.

### BRIEF DESCRIPTION OF THE FIGURES

Other characteristics and advantages of the invention will emerge from the following description, which is purely illustrative and non-limiting, and must be read in conjunction with the attached figures, of which:

FIG. 1 schematically illustrates equipment involved in a first embodiment of the invention, in which the heart of a patient is subjected to rapid cardiac stimulation;

FIG. 2 is a diagram schematically illustrating variations in electrocardiogram and blood pressure signals during rapid cardiac stimulation;

FIG. 3 is a chronological diagram schematically illustrating the succession of different steps of the image-acquisition process used by a control unit;

FIG. 4 schematically illustrates equipment involved in a second embodiment of the invention, in which the heart of a patient is subjected to an injection of a cardiac blocking solution;

FIG. 5 is a diagram illustrating steps of an image-acquisition process, in keeping with the first embodiment of the invention;

FIG. 6 is a diagram illustrating steps of an image-acquisition process, in keeping with the second embodiment of the invention;

FIG. 7 is a diagram illustrating instances of interruption of the processes of FIGS. 5 and 6.

### DETAILED DESCRIPTION

In FIG. 1, a system in keeping with a first embodiment comprises a monitoring device of the patient 1, an image acquisition device 2, a control unit 3 of the image acquisition device, a respirator 4, a cardiac stimulator 5, a control unit 6 of the respirator and of the cardiac stimulation device, an injection device of contrast product and an activator 7 for triggering the system. [fast stimulation is made by the cardiac pacemaker—it is the same apparatus]

The monitoring device 1 comprises sensors to be arranged on or inside the patient for measuring physiological parameters of the patient, especially an electrocardiogram (ECG) and blood pressure, as well as an interface unit for registering and displaying the measuring signals.

The image acquisition device 2 comprises a C-shaped arm, a source of X-rays and a detector, the source and the detector each being fixed to an end of the C-shaped arm. The imaging device 2 acquires a sequence of projective images of the heart of a patient lying prone on an operating table.

The control unit 3 of the image acquisition device is programmed to execute a process of image acquisition, such as illustrated in FIG. 5. This process synchronises the image acquisition device 2, the respirator 4, the cardiac stimulator 5 and the injection device of the contrast product. This synchronisation limits the dose of X-rays received by the patient and limits the dose of contrast product administered to the patient.

The respirator 4 is a mechanical ventilation device which compensates for the spontaneous respiration of the patient when the patient is under the effect of anaesthesia.

The cardiac stimulator 5 comprises an electric impulse generator and electrodes to be placed in the heart of the patient for transmitting the electric impulses to the cardiac cells. The cardiac stimulator 5 artificially modifies the cardiac frequency of the heart.

The injection device 9 performs an intra-arterial injection of a contrast product (such as an iodine composition) opaque to X-rays. The contrast product brings out the vascular network on the images acquired by the image acquisition device.

The triggering activator 7 allows an operator to trigger or stop the process. The activator 7 can for example comprise an interrupter which can be activated manually or by a pedal in turn activated by foot by the operator.

FIG. 5 is a diagram illustrating steps of a process for image acquisition, in keeping with the first embodiment of the invention.

At the outset of the process it is assumed that the imaging device is in a state 50 in which rotation of the C-shaped arm is authorised. Typically, the C-shaped arm is previously driven in rotation to describe a test movement to verify that no obstacle is impeding movement of the acquisition device.

According to a first step 51, the operator activates the triggering activator. For example, the operator presses on the pedal. The triggering activator transmits a triggering signal to the control unit.

According to a second step 52, the control unit transmits to the control unit of the cardiac stimulator a stimulation start signal. In response to this signal, the control unit controls rapid stimulation of the heart. The heart is typically stimulated with electric signals having a high stimulation frequency, of the order of 200 pulses per minute, the effect of which is to stop the movement of the blood in the heart and lower blood pressure.

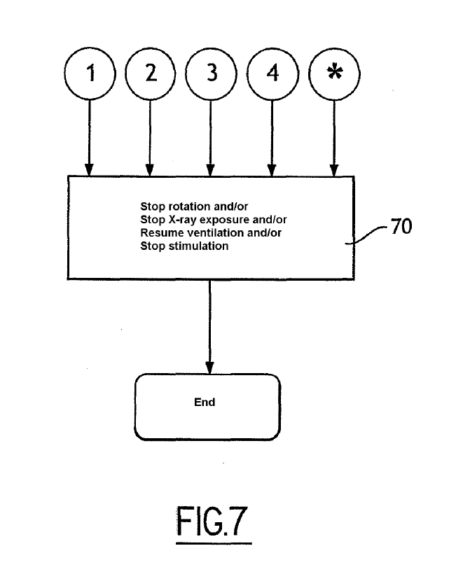

According to a third step 53, the control unit determines whether cardiac stimulation is satisfactory. To this effect, the control unit compares the frequency of the electrocardiogram measured to the frequency of the stimulation signals. If the frequency measured corresponds to the stimulation frequency or to a submultiple of the stimulation frequency as a function of a desired capture rate, then the control unit executes a fourth step 54. In the opposite case, the control unit executes step 70 (FIG. 7).

The term “capture” designates the reaction of the heart to artificial stimulation. The rate of capture is the ration between the measured cardiac frequency and frequency of the stimulation signals.

In general, in the present process, the desired capture rate is 1:1. If the capture rate is 1:2 (1 heart beat per 2 stimuli) at a certain stimulation frequency (for example 200 Hz), the control unit controls the cardiac stimulator to modify the characteristics of the stimulation signal (duration, amplitude) or lower the frequency (for example 180 Hz) to obtain a capture rate de 1:1. For example, the desired cardiac frequency is 200 beats per minute (capture rate of 1:1), the measured cardiac frequency is at first 100 bpm (capture rate of 1:2), then after adjustment of the stimulation signal, the measured cardiac frequency is 180 bpm.

An adjustment technique of the stimulation frequency is described for example in the publication: <<Rapid pacing to facilitate transcatheter prosthetic heart valve implantation”, John G. Webb, Sandjeevan Pasupati, Leslie Achtem, Christopher R. Thompson, Catherization and Cardiovascular Interventions 68:199-204 (2006). The technique described in this publication in the context of an implantation procedure for a heart valve consists of decreasing the stimulation frequency in increments of 10 to 20 bpm until the capture is correct.

According to a fourth step 54, the control unit compares the blood pressure measured to a value of reference blood pressure. If the blood pressure measured is less than the reference pressure, then the control unit executes a fifth step 55. In the opposite case, the control unit executes step 70 (FIG. 7).

According to a fifth step 55, the control unit transmits to the respirator a signal for stopping ventilation. In response to this signal, the ventilator is stopped, allowing the thoracic cage of the patient to be immobilised.

According to a sixth step 56, the control unit again verifies the cardiac frequency and the blood pressure measured.

If the frequency measured corresponds to the stimulation frequency or to a submultiple of the stimulation frequency and if the blood pressure measured is less than the reference pressure, then the control unit executes a seventh step 57. In the opposite case, the control unit executes step 70 (FIG. 7).

According to a seventh step 57, the control unit transmits to the injection device an injection start signal. In response to this signal, the injection device commences injecting the contrast product.

According to an eighth step 58, the control unit measures the time elapsing from the start of injection. During this time, the injected contrast product spreads in the blood. At the end of a predetermined period, the control unit executes a ninth step 59. The duration of the predetermined period is fixed to allow adequate diffusion of the contrast product in the blood prior to launching acquisition.

According to the ninth step 59, the control unit transmits to the acquisition device a rotation start signal. In response to this signal, the acquisition device drives the C-shaped arm in rotation.

According to a tenth step 510, the control unit transmits to the imaging device a signal for triggering acquisition. In response to this signal, the image acquisition device continues rotation of the C-shaped arm and acquires a sequence of projected images. To this effect, the source emits radiation which is transmitted to the detector via the body of the patient. When acquisition is complete, the control unit executes an eleventh step 511. The sequence of images acquired is transmitted to the control unit and recorded in memory. The sequence of projective images could be processed later to generate a three-dimensional image of the heart from the projected images.

The injection of the contrast product is continued throughout steps 58 to 510.

According to the eleventh step 511, the control unit transmits to the image acquisition device a signal for end of acquisition. The acquisition device stops rotation of the C-shaped arm and emission of the radiation.

The control unit transmits to the respirator a signal to resume ventilation, such that the respirator restores the ventilation of the patient.

The control unit transmits to the cardiac stimulation device a signal for stopping stimulation, such that the stimulation device stops the rapid stimulation of the heart. The cardiac frequency resumes a normal frequency and the blood pressure is regained.

The control unit transmits to the injection device a signal for stopping the injection, such that the injection device ceases injecting the contrast product.

Throughout steps 50 to 511, the operator must maintain pressure on the triggering activator. If the operator relinquishes pressure on the activator, the entire process is interrupted, and the control unit executes step 70 (FIG. 7).

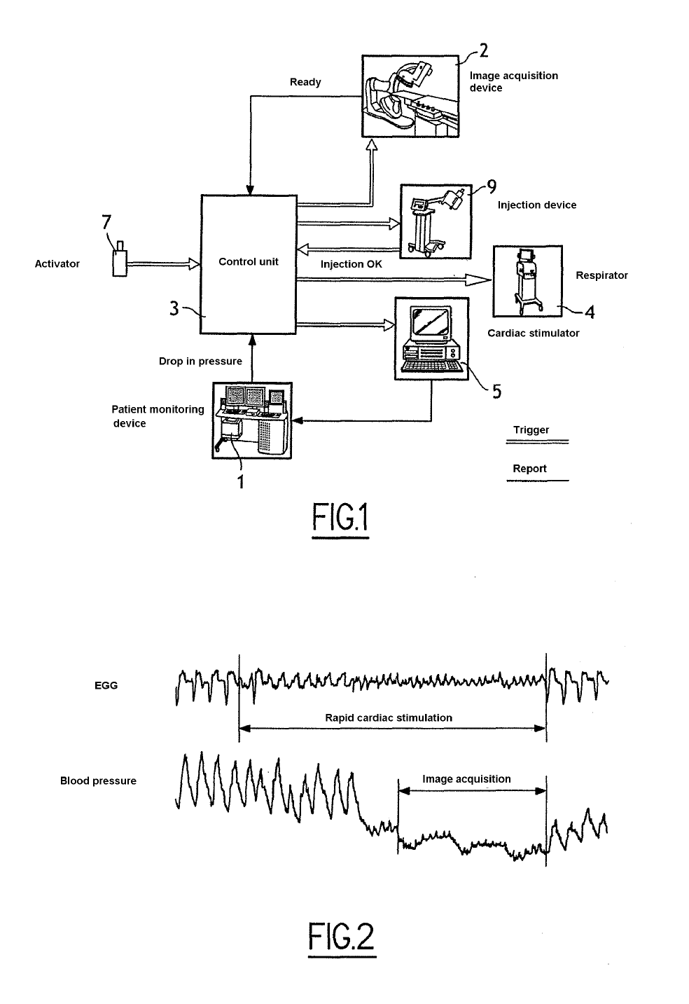

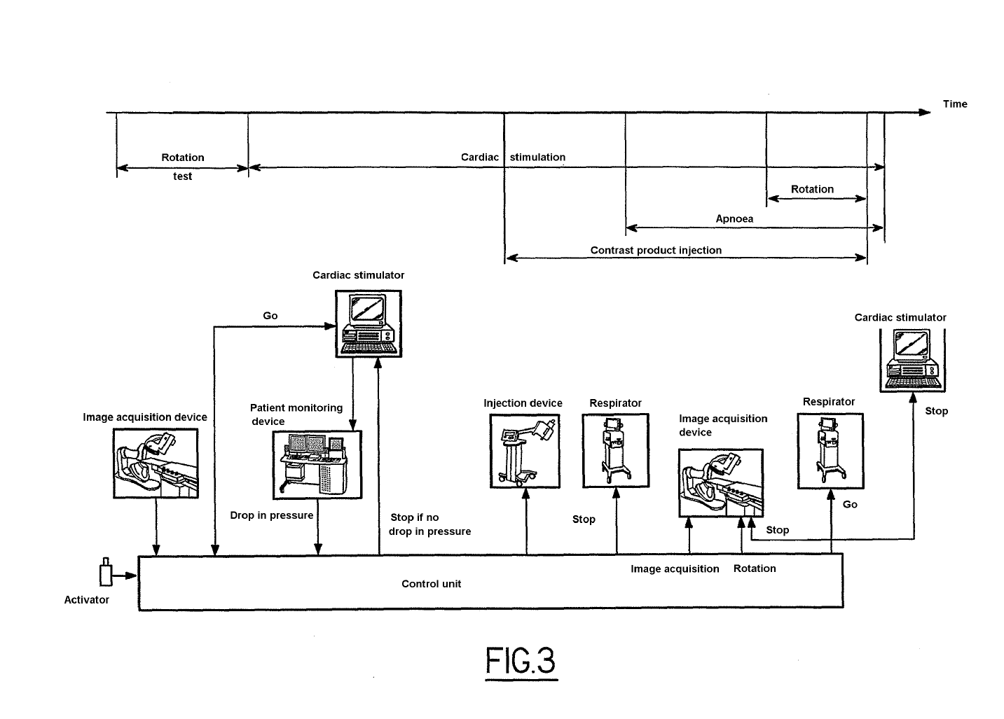

FIG. 2 is a diagram schematically illustrating variations in electrocardiogram and blood pressure signals during rapid cardiac stimulation.

The acquisition (steps 57 to 510) of the sequence of images is launched only when cardiac stimulation is satisfactory, that is, when the frequency measured corresponds to the stimulation frequency or to a submultiple of the stimulation frequency and when the blood pressure measured is less than the reference pressure.

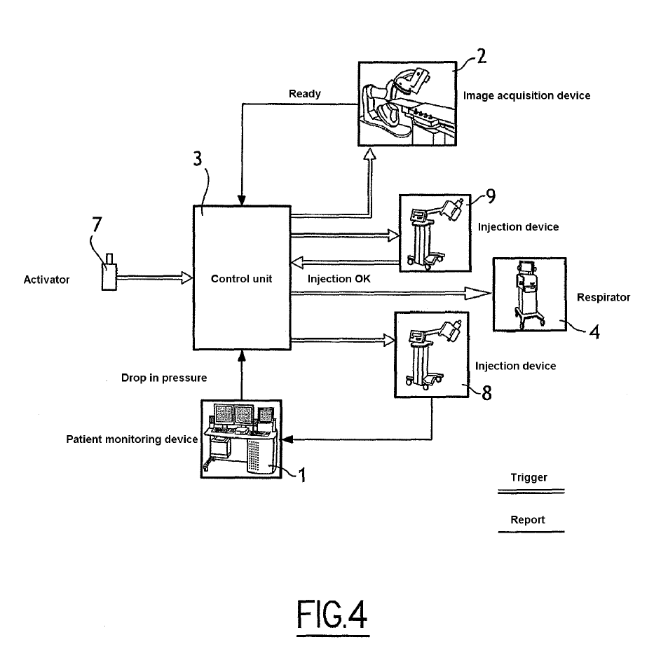

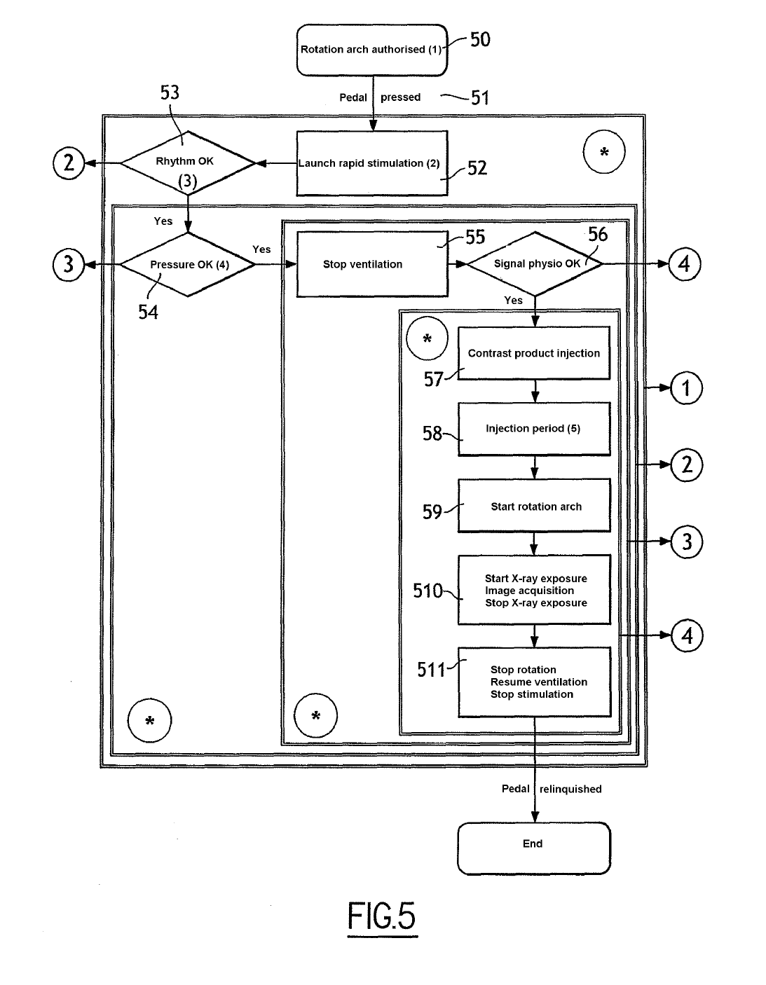

In FIG. 4, a system in keeping with a second embodiment comprises equipment identical to the equipment of FIG. 1, specifically: a monitoring device of the patient 1, an image acquisition device 2, a control unit 3 of the image acquisition device, a respirator 4, an injection device for contrast product 9 and a triggering activator 7 of the system.

However, in this second embodiment, the system comprises no cardiac stimulator, rather an injection device of a cardiac blocking solution 8 (such as a solution of adenosine triphosphate).

Also, the control unit 6 is able to control the respirator 4 and the injection device 8.

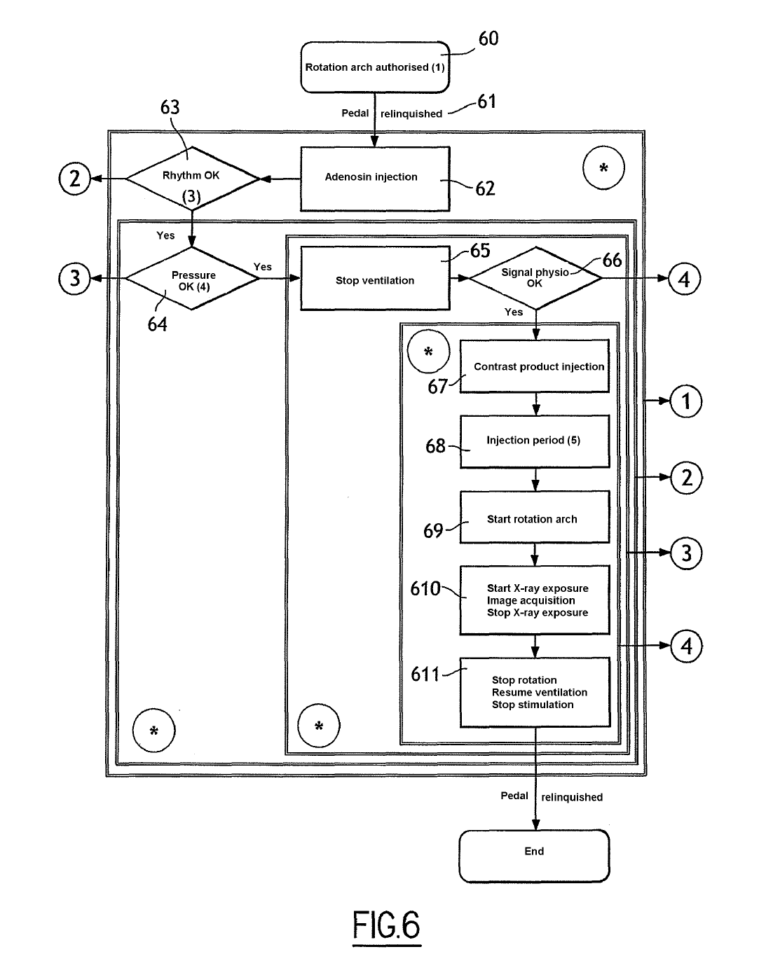

FIG. 6 is a diagram illustrating steps of a process for image acquisition, in keeping with the second embodiment of the invention.

The process comprises steps 61 and 63 to 611, identical to steps 51 and 53 to 511 of FIG. 5, except that step 52 of FIG. 5 is replaced by a step 62 in FIG. 6.

According to this second step 62, the control unit transmits to the control unit of the injection device for cardiac blocking solution an injection start signal. In response to this signal, the control unit controls injection of the blocking solution. The effect of the blocking solution is to immobilise the movement of the heart and consequently to stop the movement of the blood in the heart and lower blood pressure.

In the first and the second embodiment (FIGS. 5 and 6), the control unit constantly receives signals for measuring electrocardiogram and blood pressure. As soon as the control unit detects an anomaly in the evolution of these signals (slowing of the cardiac frequency or rise of the blood pressure), the control unit interrupts the process.

FIG. 7 is a diagram illustrating instances of interruption to the processes of FIGS. 5 and 6.

Case (1) corresponds to the case where the operator ceases exerting pressure on the triggering activator. Therefore, the operator can interrupt the process at any time and bring the patient back to his initial state.

Case (2) corresponds to the case where cardiac frequency does not correspond to the stimulation frequency of the cardiac stimulation device (in the first embodiment) or is greater than the expected cardiac frequency (in the second embodiment).

Case (3) corresponds to the case where the blood pressure is greater than the reference pressure.

Cases (2) and (3) occur when the heart is not in a reduced satisfactory state of movement to carry out acquisition of radiological images.

Case (4) corresponds to the case where other measured physiological parameters (such as arterial pressure) are not considered as satisfactory. For example, systolic pressure is less than 60 mmHg (millimetres of mercury) and diastolic pressure is under 15 mmHg.

Case (*) corresponds to other possible cases requiring interruption of the process, such as for example instances of errors in the execution of the program or exceeding of timeout.

What is claimed is:

1. A process for acquiring a three-dimensional radiological image of an organ in movement of a patient, according to which a control unit executes steps of:
(53, 54, 63, 64) receiving a signal representative of a movement parameter of the organ,
(53, 54, 63, 64) detecting variation in the signal due to artificial maintenance of the organ in a reduced state of movement, and
in response to detection, (59, 510, 69, 610) triggering acquisition of a sequence of images by a radiological imaging device to reconstruct a three-dimensional radiological image of the organ from the sequence of images.

2. The process as claimed in claim 1, in which the detection step comprises comparing the received signal with an expected reference signal indicative of a state of reduced movement of the organ.

3. The process as claimed in claim 1, further comprising a step of:
(52, 62) controlling maintenance of the organ in a reduced state of movement.

4. The process as claimed in claim 3, in which maintenance of the organ in a reduced state of movement is realised by rapid cardiac stimulation (52).

5. The process as claimed in claim 3, in which maintenance of the organ in a reduced state of movement is realise by injection of a blocking solution (62).

6. The process as claimed in claim 1, further comprising a step of:
(55, 65) controlling stopping a ventilation device of the patient prior to triggering the acquisition step.

7. The process as claimed in claim 1, further comprising steps of:
(57, 67) controlling injection of a contrast product,
(58, 68) timing a period for diffusion of the contrast product and (59, 69) triggering acquisition of a sequence of images once the period has elapsed.

8. The process as claimed in claim 1, further comprising a step of:
(56, 66) monitoring evolution of physiological parameters of the patient,
(70) controlling interruption of the process in the event of detection of abnormal evolution of one of the physiological parameters.

9. The process as claimed in claim 1, further comprising steps of:
(51, 61) detecting pressure exerted on a triggering activator by an operator,
(70) controlling interruption of the process in the event of relinquishing pressure exerted by the operator.

10. The process as claimed in claim 8, in which the interruption step (70) of the process comprises the ceasing to maintain the organ in a reduced state of movement.

11. A control unit of an acquisition device of a three-dimensional radiological image, the unit being suitable for receiving at the input a signal representative of a movement parameter of an organ and generating at the output a signal for controlling the image acquisition device, the unit being programmed to execute the steps defined by claim 1.

12. A radiological imaging device comprising a control unit as claimed in claim 11.
